(19) United States
(12) Patent Application Publication
(10) Pub. No.: US 2025/0378916 A1
(43) Pub. Date: Dec. 11, 2025

(54) VIRAL ESCAPE INSPIRED FRAMEWORK FOR PRECISION STRUCTURE-GUIDED DUAL BAIT PROTEIN BIOSENSOR DEVELOPMENT

(71) Applicant: Iowa State University Research Foundation, Inc., Ames, IA (US)

(72) Inventors: Ratul Chowdhury, Ames, IA (US); Mohammed Sakib Noor, Ames, IA (US)

(21) Appl. No.: 19/229,639

(22) Filed: Jun. 5, 2025

Related U.S. Application Data
(60) Provisional application No. 63/656,226, filed on Jun. 5, 2024.

(51) Int. Cl. G16B 40/20; G16B 15/30; G16B 20/20; G16B 35/10

(52) U.S. Cl. CPC G16B 40/20; G16B 15/30; G16B 20/20; G16B 35/10

(57) ABSTRACT

Platforms, systems, and methods for the simulation and identification of future escape variants of viruses that via a protein receptor on the host and/or dual bait biosensors are provided. The platforms and workflows leverage computer tools and artificial intelligence to quickly and reliably identify future escape variants and proteins optimal for dual bait biosensors, thereby reducing the lead time of vaccine development and allowing for preemptive and predictive antibody design.

### CROSS REFERENCE TO RELATED APPLICATIONS

This application claims priority under 35 U.S.C. § 119(e) to provisional patent application U.S. Ser. No. 63/656,226, filed Jun. 5, 2024. The provisional patent application is hereby incorporated by reference in its entirety herein, including without limitation: the specification, claims, and abstract, as well as any figures, tables, appendices, or drawings thereof.

### TECHNICAL FIELD

The present disclosure relates generally to systems and methods of predicting and/or identifying potential escape variants of viruses to aid in the preemptive design of antibodies and vaccines.

### BACKGROUND

There is a significant industrial demand for selective biosensors in fields like environmental monitoring, healthcare diagnostics, food safety, and green energy production. Specifically, detecting specific (heavy metal, toxin) contaminants in water or identifying disease biomarkers requires biosensors with high specificity and sensitivity. Rapid design is crucial to meet these needs, especially in responding to emerging challenges.

Viruses can have devastating public health and food supply consequences. For example, SARS-CoV-2 has infected over 700 million individuals, and the death toll has reached 7 million worldwide, while in the USA, a total of 1.2 million lives have been lost to the pandemic. While the health sector was collapsing, the countrywide lockdown caused economies to falter, with the USA experiencing its highest unemployment rate since 1930. Traditional methods of controlling viruses, such as vaccination, can be effective but are not always future-proof as viruses mutate over time to resist vaccines. At the onset of the SARS-CoV-2 pandemic, the virus was gaining approximately two mutations a month in the global population, and since then, the World Health Organization (WHO) has recognized 11 critical variants of SARS-CoV-2. These variants caused a drop in effectiveness of vaccines. Therefore, it is key to anticipate viral escape variants with enough lead time. Currently, there is a lack of technology for viral mutation prediction.

Thus, there is a need in the art for methods and systems for predicting prospective viral escape variants to allow for the development of biosensors that remain effective against future viral variants.

### SUMMARY

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage to address previous challenges of reliably, efficiently, and quickly identifying future viral escape variants associated with viruses.

It is a further object, feature, and/or advantage to provide a platform that allows for modularity in the choice of tools for (a) antigen sequence prediction, (b) antigen structure prediction, (c) docking, and (d) computational scoring of binding affinity.

Modular platforms for use in identifying escape variants are provided. In some embodiments, the platforms comprise a sequence optimization module comprising a sequence design module; and a structure tracking module comprising a protein docking module and/or a structure prediction module.

Methods for identifying escape variants are also provided. In some embodiments, the methods comprise identifying an amino acid interaction in a protein-protein complex between a first and second protein and, optionally, between a first and third protein; identifying at least one mutation in the first, second, and/or third protein that would disrupt the amino acid interaction; ranking the at least one mutation and selecting at least one favorable mutation; updating the amino acid interaction of step (a) with the favorable mutation to generate a new amino acid interaction and repeating steps (a) through (c) at least once; and generating a library of escape variants.

In other embodiments, methods of identifying escape variants comprise determining an interface distance matrix of a protein-protein complex between a first and second protein; predicting a mutated protein sequence for at least the first protein using the interface distance matrix; predicting the three dimensional structure of the mutated protein sequence; predicting docking poses of the mutated protein sequence to the second protein to generate a new interface distance matrix; and generating a library of escape variants.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Several embodiments in which the present disclosure can be practiced are illustrated and described in detail, wherein like reference characters represent like components throughout the several views. The drawings are presented for exemplary purposes and may not be to scale unless otherwise indicated.

FIG. 1A shows the overall structure of the trimeric spike protein of SARS-CoV-2 with the receptor binding domain (RBD) highlighted inset.

FIG. 1B shows Interaction interface between RBD with commercial antibody LY-CoV1404. Insets illustrate the interacting residues from the RBD and heavy (blue) and light (green) chains of the antibody proteins within 4 Å.

FIG. 2 shows an exemplary workflow according to aspects of the disclosure. CTRL-V version 1 workflow architecture with the modular elements included in the iterative loop. The leftmost box represents the structure tracking module where updated antigen (and optionally antibody) structures are predicted, and antigen-antibody docking is performed. The right box represents the sequence optimization module where the new sequences (for antigen and antibody) are predicted and evaluated.

FIG. 3A shows SARS-CoV-2 mutations and their frequency of appearance, and chemical types of point mutations in all circulating variants compared to prediction from the CTRL-V simulations. Blue represents positive, red negative, green polar, and pink hydrophobic amino acid transitions to the spike proteins. The bottom panel show those point mutations which were correctly identified across different CTRL-V simulations.

FIG. 3B shows Illustrative insight into the favored mutations in circulating infective variants across their amino acid chemical categories show CTRL-V can successfully replicate the variety of point mutations: positive, polar, and hydrophobic.

FIG. 3C shows Distribution of how far each mutation is from the antigen-antibody binding interface, its frequency across circulating variants, its binding affinity towards the ACE2 receptor, and how well it is expressed on the viral surface for each of the 39 mutations (from an experimental deep mutational scan study). CTRL-V-recovered mutations are labeled.

FIG. 4A shows performance of CTRL-version 1, including that ESMfold predicts mutated viral spike RBD shows worsening of prediction quality due to paucity of viral proteins in training data. The RMSD plot shows, worse structures get carried through to newer generations and never get fixed (i.e., maintain ˜24 Å RMSD with starting structure).

FIG. 4B shows the maximized objective function in the integer optimization model on all possible single-point mutations for viral protein at the interface. There are two distinct horizontal swaths (indicated with white text): a high preference for proline and a low preference for cysteine.

FIG. 4C shows interaction of wildtype SARS-CoV-2 spike RBD with its human entry receptor protein ACE2.

FIG. 4D shows CTRL-V version 1 captures only one true, circulating single-point mutation, V445P, out of 39 known point mutations (likely because version 1 generates a high number of proline variants).

FIG. 5 shows expression and binding score for all 39 SARS-CoV-2 single-point mutations. Experimental data was reported from deep mutational scan study by Greaney et al., Escape mutations predicted by different versions of CTRL-V are shown in pink. Neutral binding affinity and moderate expression score are more popular choices for circulating variants. CTRL-V captures the topology of the spread-out landscape by recovering at least one variant within 0.5 log₁₀ binding free energy with human ACE2 receptor protein, and 1 log₁₀ expression of any known circulating variant.

FIG. 6 shows the difference in binding energy between antigen-antibody complex and antigen-receptor complex; where antigen refers to SARS-CoV-2 spike protein, antibody is LYCoV-1404 neutralizing antibody, and receptor is human ACE2 protein. Left bar plot list all the mutations and corresponding difference in the binding energy of antigen-antibody complex over the antigen-receptor complex. Green stands for mutations in circulating variants, blue and red for (two differently posed objective functions for sequence design using) CTRL-V version 3. The first implementation (blue) identifies point mutations in spike which are stabilizing to the spike and improve binding to both the receptor and antibody, while the second implementation (red) improves binding to receptor and lowers binding with the antibody and is destabilizing for the antigen itself. Insets illustrate the key interacting residues at the interfaces of antigen (red)—receptor (yellow) and antigen (red)—antibody (blue) complexes.

FIG. 7A shows biophysical characterization of the KP.2 variant showing the five point mutations that CTRL-V deems to be causal for this variant to escape the immunity of LY-CoV1404 commercial antibody. S371F and L373P mutations on the spike introduce a hydrophobic patch close to a hydrophilic sub-surface of the antibody thereby allowing the spike to lower its affinity for the antibody and thereby escape.

FIG. 7B shows V445H and G446L mutations together introduce a similar incompatible electrostatic surface through the insertion of polar groups close to hydrophobic ones and vice versa, respectively.

FIG. 7C shows N440K mutation does not compromise the electrostatic microenvironment with the Ser103 side chain of the antibody but weakens interaction strength due to steric hindrance posed by the larger Lys440 side chain.

FIG. 8 shows hardware specifications used in the SARS-CoV-2 benchmarking study. When run for longer, i.e., more generations of variants, CTRL-V version 3 can capture more true positives escape mutations, albeit at the cost of higher false positives. The boxes to the right of the graph refer the choice of modular elements of CTRL-V for this hardware benchmarking task.

An artisan of ordinary skill in the art need not view, within isolated figure(s), the near infinite distinct combinations of features described in the following detailed description to facilitate an understanding of the present disclosure.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. The definitions are provided to aid in describing particular embodiments and are not intended to limit the claimed disclosure. Many methods and materials similar, modified, or equivalent to those described herein can be used in the practice of the embodiments without undue experimentation, but the preferred materials and methods are described herein. In describing and claiming the embodiments, the following terminology will be used in accordance with the definitions set out below.

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form. Numeric ranges recited within the specification are inclusive of the numbers within the defined range. Throughout this disclosure, various aspects are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range (e.g., 1 to 5 includes 1, 1.5, 2, 2.75, 3, 3.80, 4, and 5).

As used herein, the term “and/or”, e.g., “X and/or Y” shall be understood to mean either “X and Y” or “X or Y” and shall be taken to provide explicit support for both meanings or for either meaning, e.g., A and/or B includes the options i) A, ii) B or iii) A and B.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

The term “about,” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

“Antibodies” refers to polyclonal and monoclonal antibodies, chimeric, and single chain antibodies, as well as Fab fragments, including the products of a Fab or other immunoglobulin expression library. With respect to antibodies, the term, “immunologically specific” refers to antibodies that bind to one or more epitopes of a protein of interest, but which do not substantially recognize and bind other molecules in a sample containing a mixed population of antigenic biological molecules.

The terms “include” and “including” when used in reference to a list of materials refer to but are not limited to the materials so listed.

The term “weight percent,” “wt. %,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt. %,” etc.

The methods and compositions may comprise, consist essentially of, or consist of the components and ingredients as well as other ingredients described herein. As used herein, “consisting essentially of” means that the methods and compositions may include additional steps, components or ingredients, but only if the additional steps, components or ingredients do not materially alter the basic and novel characteristics of the claimed methods and compositions.

Aspects and/or embodiments of the present disclosure aim to overcome and/or improve on issues and challenges raised. At least one goal is to leverage artificial intelligence and/or machine learning to reliably, efficiently, and quickly identify future viral escape variants. In an aspect, this allows for the development and biomanufacturing of rapid cross-neutralizing antibodies that will remain effective against future variants.

Utilizing computational algorithms, platforms and workflows of the present disclosure can analyze the interactions between viral proteins, antibody proteins, and host receptor proteins. These analyses can reveal the most favorable mutations for viral proteins that allow the virus to (1) escape the antibody and (2) maintain binding and entry into the host. This can allow for the a priori design of broadly neutralizing antibodies that remain effective against future escape variants, thus enhancing pandemic preparedness and response capabilities. This foresight is critical for maintaining effective countermeasures against emerging viral threats, ensuring that public health responses can be swift and targeted.

Platforms and workflows of the present disclosure are modular, allowing for the substitution, addition, and/or deletion of modules based on the intended use and desired output. This modularity allows the user to leverage any other tool for the relevant steps including—(a) sequence design, (b) structure prediction, (c) docking, (d) sequence evaluation through energetics, and (e) acceptance and rejection criterion of a design.

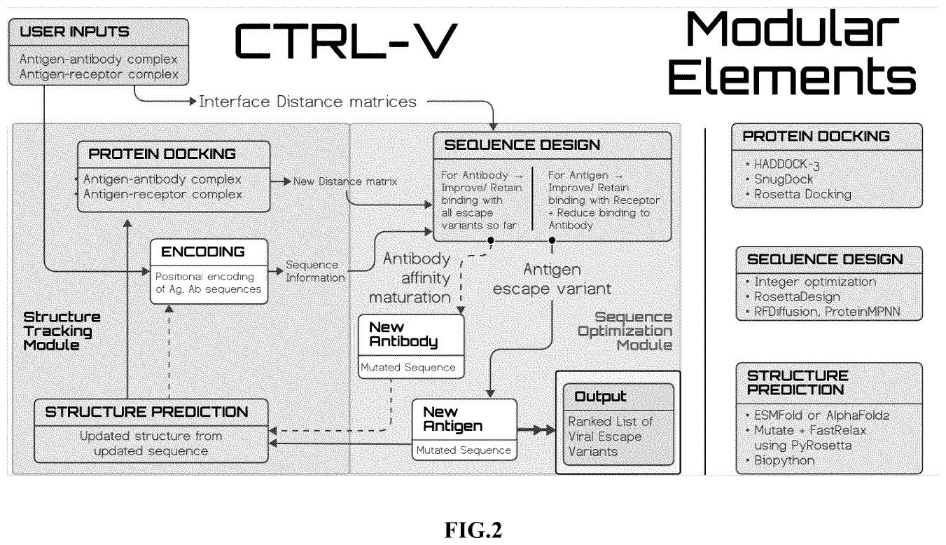

In some embodiments, the platform comprises a sequence optimization module and a structure tracking module. One embodiment is shown in FIG. 2. The sequence optimization module can comprise a sequence design module. The sequence design module can use tools such as integer optimization, RosettaDesign, RFDiffusion, and/or ProteinMPNN. It should be understood that sequence design is not limited to the tools recited herein, but rather any tool or method known in the art for sequence design may be used. In some embodiments, multiple tools are used.

The structure tracking module can comprise a protein docking module and/or a structure prediction module. The protein docking module can use tools such as HADDOCK-3, SnugDock, and/or Rosetta Docking. The structure prediction module can use tools such as ESMFold, AlphaFold2, PyRosetta, and/or Biopython. It should be understood that protein docking and structure prediction are not limited to the tools recited herein, but rather any tool or method known in the art for predicting protein docking and protein structure may be used. In some embodiments, multiple tools are used.

In some embodiments, methods of identifying escape variants comprise identifying an amino acid interaction between proteins. As used herein, “amino acid interaction” can be defined as the manner in which the amino acid residues of two or more proteins interact with each other, which may determine how the proteins dock and bind to each other. Amino acids exhibit interaction preferences with each other based on amino acid type, their secondary structure, and the contact based environment that they find themselves in the native state structure as measured by their number of neighbors. Amino acids can be assigned pairwise interaction scores based on these preferences, as fully tabulated and described by Jha et al. (Amino acid interaction preferences in proteins. Protein Sci. 2010 March; 19(3):603-16.), which is herein incorporated by reference for this purpose. An integer optimization model utilizes this preference score for sequence design, i.e., identifying point mutations that allude to the objective of improving binding to the receptor simultaneously alleviating binding with the antibody (See Example 1).

Based on the amino acid interactions, mutations can be identified that would disrupt the amino acid interactions between proteins. In some embodiments, mutation prediction can be performed using integer optimization, RosettaDesign, RFDiffusion, and/or ProteinMPNN. In some embodiments, mutation identification comprises generating a library of sequence predictions for the proteins and comparing the sequences to identify mutations.

In some embodiments, identified mutations are ranked in the order of favorability. More favorable mutations can include mutations predicted to decrease the binding affinity and/or decrease the interaction between proteins as compared to binding affinity of the wild-type proteins, such as between an antigen and antibody. More favorable mutations can also include mutations predicted to maintain or increase the binding affinity and/or interaction between proteins, such as between an antigen and a host receptor protein. In some embodiments, the method prioritizes mutations that are predicted to decrease the binding affinity of an antigen to an antibody and/or deprioritizes mutations that would decrease the binding affinity of an antigen to a host receptor protein. In this way, viral escape variants which maintain entry into the host can be simulated and predicted. Similarly, affinity maturation of an antibody can also be taken into account and predicted.

In some embodiments, the most favorable mutations are selected and used to update the amino acid sequences of the proteins in the protein-protein complexes (i.e., a new amino acid interaction). The mutation prediction, ranking, and selection steps can then be repeated for 1, 2, 3, 4, 5, 10, 20, 75, 50, 100, 1,000, or more times to generate a library of refined escape variants. In some embodiments, the library of escape variants comprises a list of predicted single-point mutations.

Methods of the disclosure can leverage a variety of tools to make predictions and rank lists, including, for example, RosettaDesign, RFDiffusion, ProteinMPNN, ESMFold, AlphaFold2, PyRosetta, Biopython, HADDOCK-3, SnugDock, Rosetta Docking, and the like. It should be understood that the methods are not limited to the tools recited herein, but rather any tool or method known in the art may be used. In some embodiments, multiple tools are used. In some embodiments, artificial intelligence and/or machine learning is used.

Methods, platforms, and workflows described herein are not limited to the prediction of viral escape variants. The scope and utility can also be used for designing peptide-based discriminatory biosensors, small molecules, and even metal and non-metal ions.

### Software, Computer System, and Network Environment

Some embodiments described herein make use of computer algorithms in the form of software instructions executed by a computer processor. In some embodiments, the software instructions include a machine learning module, also referred to herein as artificial intelligence software. As used herein, a machine learning module refers to a computer implemented process (e.g., a software function) that implements one or more specific machine learning algorithms, such as an artificial neural network (ANN), convolutional neural network (CNN), random forest, decision trees, support vector machines, and the like, in order to determine, for a given input, one or more output values. In some embodiments, the input comprises alphanumeric data which can include numbers, words, phrases, or lengthier strings, for example. In some embodiments, the one or more output values comprise values representing numeric values, words, phrases, or other alphanumeric strings. In some embodiments, the one or more output values comprise an identification of one or more response strings (e.g., selected from a database).

For example, a machine learning module may receive as input a textual string (e.g., entered by a human user, for example) and generate various outputs. For example, the machine learning module may automatically analyze the input alphanumeric string(s) to determine output values classifying a content of the text (e.g., an intent).

In some embodiments, machine learning modules implementing machine learning techniques are trained, for example using datasets that include categories of data described herein. Such training may be used to determine various parameters of machine learning algorithms implemented by a machine learning module, such as weights associated with layers in neural networks. In some embodiments, once a machine learning module is trained, e.g., to accomplish a specific task such as identifying certain response strings, values of determined parameters are fixed and the (e.g., unchanging, static) machine learning module is used to process new data (e.g., different from the training data) and accomplish its trained task without further updates to its parameters (e.g., the machine learning module does not receive feedback and/or updates). In some embodiments, machine learning modules may receive feedback, e.g., based on user review of accuracy, and such feedback may be used as additional training data, to dynamically update the machine learning module. In some embodiments, two or more machine learning modules may be combined and implemented as a single module and/or a single software application. In some embodiments, two or more machine learning modules may also be implemented separately, e.g., as separate software applications. A machine learning module may be software and/or hardware. For example, a machine learning module may be implemented entirely as software, or certain functions of an ANN module may be carried out via specialized hardware (e.g., via an application specific integrated circuit (ASIC)).

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, some modules described herein can be separated, combined or incorporated into single or combined modules. Any modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

While the methods and systems of present disclosure has been particularly shown and described with reference to specific preferred embodiments, it should be understood that various changes in form and detail may be made therein without departing from the spirit and scope of the present disclosure.

### AI/Machine Learning

Many statistical classification techniques are suitable as approaches to perform the classification described herein. Such methods include but are not limited to supervised learning approaches.

Commonly used supervised classifiers include without limitation the neural network (e.g., artificial neural network, multi-layer perceptron), support vector machines, k-nearest neighbors, Gaussian mixture model, Gaussian, naive Bayes, decision tree and radial basis function (RBF) classifiers. Linear classification methods include Fisher's linear discriminant, logistic regression, naive Bayes classifier, perceptron, and support vector machines (SVMs). Other classifiers for use with methods according to the disclosure include quadratic classifiers, k-nearest neighbor, boosting, decision trees, random forests, neural networks, pattern recognition, Bayesian networks and Hidden Markov models. Other classifiers, including improvements or combinations of any of these, commonly used for supervised learning, can also be suitable for use with the methods described herein.

Classification using supervised methods can generally be performed by the following methodology:
1. Gather a training set. The training samples are used to “train” the classifier.
2. Determine the input “feature” representation of the learned function. The accuracy of the learned function depends on how the input object is represented. Typically, the input object is transformed into a feature vector, which contains a number of features that are descriptive of the object. The features may include clinical features of a patient or subject.
3. Determine the structure of the learned function and corresponding learning algorithm. A learning algorithm is chosen, e.g., artificial neural networks, decision trees, Bayes classifiers or support vector machines. The learning algorithm is used to build the classifier.
4. Build the classifier (e.g., classification model). The learning algorithm is run on the gathered training set. Parameters of the learning algorithm may be adjusted by optimizing performance on a subset (called a validation set) of the training set, or via cross-validation. After parameter adjustment and learning, the performance of the algorithm may be measured on a test set of naive samples that is separate from the training set. The built model can involve feature coefficients or importance measures assigned to individual features.

In some cases, the individual features are clinical features. In some cases, the clinical feature is a normalized value, an average value, a median value, a mean value, an adjusted average, or other adjusted level or value.

Once the classifier (e.g., classification model) is determined as described above (“trained”), it can be used to classify a sample, e.g., clinical features that are analyzed or processed according to methods described herein.

The trained model and the associated machine learning and application of the model will utilize processors, modules, memories, databases, networks, and potentially user interfaces to show the results and allow changes to be made.

In communications and computing, a computer readable medium is a medium capable of storing data in a format readable by a mechanical device. The term “non-transitory” is used herein to refer to computer readable media (“CRM”) that store data for short periods or in the presence of power such as a memory device.

One or more embodiments described herein can be implemented using programmatic modules, engines, or components. A programmatic module, engine, or component can include a program, a sub-routine, a portion of a program, or a software component or a hardware component capable of performing one or more stated tasks or functions. A module or component can exist on a hardware component independently of other modules or components. Alternatively, a module or component can be a shared element or process of other modules, programs, or machines.

The system will preferably include an intelligent control (i.e., a controller) and components for establishing communications. Examples of such a controller may be processing units alone or other subcomponents of computing devices. The controller can also include other components and can be implemented partially or entirely on a semiconductor (e.g., a field-programmable gate array (“FPGA”)) chip, such as a chip developed through a register transfer level (“RTL”) design process.

A processing unit, also called a processor, is an electronic circuit which performs operations on some external data source, usually memory or some other data stream. Non-limiting examples of processors include a microprocessor, a microcontroller, an arithmetic logic unit (“ALU”), and most notably, a central processing unit (“CPU”). A CPU, also called a central processor or main processor, is the electronic circuitry within a computer that carries out the instructions of a computer program by performing the basic arithmetic, logic, controlling, and input/output (“I/O”) operations specified by the instructions. Processing units are common in tablets, telephones, handheld devices, laptops, user displays, smart devices (TV, speaker, watch, etc.), and other computing devices.

The memory includes, in some embodiments, a program storage area and/or data storage area. The memory can comprise read-only memory (“ROM”, an example of non-volatile memory, meaning it does not lose data when it is not connected to a power source) or random-access memory (“RAM”, an example of volatile memory, meaning it will lose its data when not connected to a power source). Examples of volatile memory include static RAM (“SRAM”), dynamic RAM (“DRAM”), synchronous DRAM (“SDRAM”), etc. Examples of non-volatile memory include electrically erasable programmable read only memory (“EEPROM”), flash memory, hard disks, SD cards, etc. In some embodiments, the processing unit, such as a processor, a microprocessor, or a microcontroller, is connected to the memory and executes software instructions that are capable of being stored in a RAM of the memory (e.g., during execution), a ROM of the memory (e.g., on a generally permanent basis), or another non-transitory computer readable medium such as another memory or a disc.

Generally, the non-transitory computer readable medium operates under control of an operating system stored in the memory. The non-transitory computer readable medium implements a compiler which allows a software application written in a programming language such as COBOL, C++, FORTRAN, or any other known programming language to be translated into code readable by the central processing unit. After completion, the central processing unit accesses and manipulates data stored in the memory of the non-transitory computer readable medium using the relationships and logic dictated by the software application and generated using the compiler.

In one embodiment, the software application and the compiler are tangibly embodied in the computer-readable medium. When the instructions are read and executed by the non-transitory computer readable medium, the non-transitory computer readable medium performs the steps necessary to implement and/or use the present invention. A software application, operating instructions, and/or firmware (semi-permanent software programmed into read-only memory) may also be tangibly embodied in the memory and/or data communication devices, thereby making the software application a product or article of manufacture according to the present invention.

The database is a structured set of data typically held in a computer. The database, as well as data and information contained therein, need not reside in a single physical or electronic location. For example, the database may reside, at least in part, on a local storage device, in an external hard drive, on a database server connected to a network, on a cloud-based storage system, in a distributed ledger (such as those commonly used with blockchain technology), or the like.

It is envisioned that the machine learned model and any of the training of the same could include cloud computing. Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service.

A cloud computing environment is service oriented with a focus on statelessness, low coupling, modularity, and semantic interoperability. At the heart of cloud computing is an infrastructure comprising a network of interconnected nodes.

As noted, the training model could be implemented on a user interface. The interface could also be a point on introduction of data, such as training data or test data to compare to the trained model for analysis. The results of the comparison could then be shown on a user interface.

A user interface is how the user interacts with a machine. The user interface can be a digital interface, a command-line interface, a graphical user interface (“GUI”), oral interface, virtual reality interface, or any other way a user can interact with a machine (user-machine interface). For example, the user interface (“UI”) can include a combination of digital and analog input and/or output devices or any other type of UI input/output device required to achieve a desired level of control and monitoring for a device. Examples of input and/or output devices include computer mice, keyboards, touchscreens, knobs, dials, switches, buttons, speakers, microphones, LIDAR, RADAR, etc. Input(s) received from the UI can then be sent to a microcontroller to control operational aspects of a device.

The user interface module can include a display, which can act as an input and/or output device. More particularly, the display can be a liquid crystal display (“LCD”), a light-emitting diode (“LED”) display, an organic LED (“OLED”) display, an electroluminescent display (“ELD”), a surface-conduction electron emitter display (“SED”), a field-emission display (“FED”), a thin-film transistor (“TFT”) LCD, a bistable cholesteric reflective display (i.e., e-paper), etc. The user interface also can be configured with a microcontroller to display conditions or data associated with the main device in real-time or substantially real-time.

Any components of the system could be connected via network or other communication protocol to transfer information, communicate with other systems, or provide other connectivity. In some embodiments, the network is, by way of example only, a wide area network (“WAN”) such as a TCP/IP based network or a cellular network, a local area network (“LAN”), a neighborhood area network (“NAN”), a home area network (“HAN”), or a personal area network (“PAN”) employing any of a variety of communication protocols, such as Wi-Fi, Bluetooth, ZigBee, near field communication (“NFC”), etc., although other types of networks are possible and are contemplated herein. The network typically allows communication between the communications module and the central location during moments of low-quality connections. Communications through the network can be protected using one or more encryption techniques, such as those techniques provided by the Advanced Encryption Standard (AES), which superseded the Data Encryption Standard (DES), the IEEE 802.1 standard for port-based network security, pre-shared key, Extensible Authentication Protocol (“EAP”), Wired Equivalent Privacy (“WEP”), Temporal Key Integrity Protocol (“TKIP”), Wi-Fi Protected Access (“WPA”), and the like.

### EXAMPLES

### Example 1

The present disclosure provides a modular simulation platform capable of preemptively identifying and targeting potential viral escape mutations. The disclosure is demonstrated using SARS-CoV-2 as a model virus.

Respiratory viruses, such as SARS-CoV-2, enter the human/host through various molecular interaction routes at the epithelial lining of the trachea or lungs (bronchi). Viral capsid/envelope proteins (spikes) bind to protein (such as angiotensin-converting enzyme-2-ACE2 for SARS-CoV-2) or carbohydrate-based ciliary receptors (such as sialic acid for influenza-A) on host epithelia. This binding event is the first step necessary for infection and facilitates the downstream deployment of the virus's genetic material (DNA or RNA) into the cell and subsequent utilization of the host cellular machinery for replication and dissemination to other parts of the body by entering the bloodstream.

The immune system initiates a response upon detecting the presence of foreign antigenic viral proteins in an attempt to neutralize them. This involves the release of T cells and B cells, both capable of recognizing and reacting to known antigenic epitopes. T cells are responsible for eliminating infected cells, while B cells are responsible for generating antibodies. These antibodies possess the capacity to bind to antigens within the body by attaching their paratopes to the antigenic epitopes. This binding event between the antibody and antigen facilitates the recognition of antigens by phagocytotic cells, ultimately leading to their elimination. However, for novel viruses, the body fails to provide humoral resistance, and inoculation of extraneous antibodies through vaccination becomes necessary to combat the viral infection.

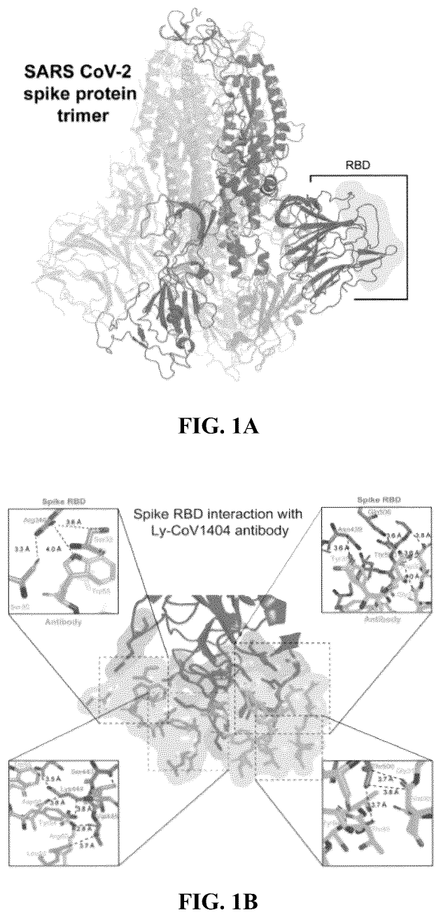

For example, SARS-CoV-2 spike protein mediates virus attachment to host cell-surface receptors (ACE2) and fusion between virus and cell membranes. This transmembrane glycoprotein consists of two subunits, S1 and S2. S1 contains the amino-terminal domain and the receptor binding domain (RBD; FIG. 1) and S2 includes the trimeric core of the protein and is responsible for membrane fusion. RBD is a highly variable region as predominant mutations occur in this domain, which allows the virus to reduce the neutralizing efficacy of the antibodies and enhance the binding with the ACE 2. Viral escape, thus, refers to mutation events in the RBD that allow for the evasion of antibodies and increased ACE2 binding. For instance, the mutation N439K first emerged in Scotland in March 2020, which was reported to result in high binding affinity with the ACE2 receptor but decreased the neutralizing effect of the sera antibodies. In the scope of this study, the landscape of single-point mutations in the RBD will be investigated based on simultaneous reduction in affinity towards a commercial antibody while maintaining/improving affinity towards the ACE2 receptor (i.e., entry protein).

### Importance of Capturing Virus Mutations

Traditional methods of controlling viruses, such as vaccination, can be effective but are not always future-proof as viruses mutate over time to resist vaccines. To this end, the presently disclosed platforms simulate the viral escape paths to identify the virus mutations that are favorable (later referred to as the Favorable Mutation Problem; FMP). An embodiment of modular platforms of the disclosure are referred to throughout the Examples as “CTRL-V” (Computational Tracking of Likely Viral Escape Variants). While the term “CTRL-V” is used herein for example purposes, it should be understood that this is a non-limiting designation of any of the systems or methods disclosed. In other words, it is a way to reference the system and method of the workflow architecture shown in FIG. 2. However, as will also be understood, the term may reference multiple variations of the workflow, as well as variations in aspects of any of the embodiments referenced herein.

Prior art methods of viral escape prediction are limited. Methods using language model-based approaches or historical viral sequences fundamentally rely on a massive corpus of sequence data, limiting their any interpretability on the physics of binding and infectivity. Consequently, they cannot be applied more broadly as a protein engineering platform to design dual bait biosensors that discriminate between different interaction partners. Current tools thus implicitly optimize a latent sequence fitness objective for viral mutation prediction and cannot provide any structural underpinning of how mutations help to discriminate between different interaction partners. Other methods using primary RNA sequence-structure alignments function, at best, as a proxy and do not necessarily translate to accurate prediction of viral protein structure. More specialized tools for antibody-antigen interactions may predict antibody-antigen binding affinity changes, but these tools ignore the interaction between antigen and receptor and have (a) an incomplete biochemical objective function, and hence (b) cannot be applied for efficient viral escape prediction or dual bait biosensor design involving multiple proteins and pairwise interactions. Beneficially, the workflow of the present disclosure can be used for both predicting viral escape and biosensor design.

CTRL-V provides structural insight to individual residue-level contribution of energetics and a strong biochemical prior for understanding why and how proteins of interest can discriminate between different interacting proteins (and potentially, metal ions, nucleotides, and small molecules). CTRL-V distinguishes itself from these existing approaches by (a) structural characterization of selection pressure on viruses, and (b) unlike tools dependent on historical data or experimental training sets, CTRL-V employs a modular, physics-based framework that integrates established computational tools to analyze protein-protein interactions without requiring prior training data.

The Examples will demonstrate that CTRL-V simulates the viral escape process of SARS-CoV-2 when confronted with commercial antibody (LY-CoV1404; constituents of the Moderna® and Pfizer® vaccines) while retaining binding with the human ACE2 receptor. CTRL-V is able to unveil a putative viral mutational landscape and identify the mutations that are most likely to enable the virus to escape from the immune response, which is critical information that can be used to develop antiviral therapies and add to global biotechnological readiness in allaying pandemics.

CTRL-V, including any of the aspects of any of the embodiments disclosed herein, also provides a new metric to rapidly, computationally assess viral escape potential of de novo designed antibodies. Even though multiple predicting techniques such as protein structure prediction and protein sequence designing have been largely explored in recent times, there has not been much work done in predicting viral mutation with the sole purpose of understanding fundamental tenets of viral mutation preferences due to the high variabilities of factors. These Examples focus on the viral mutations that happen in the viral spike protein, where predominant mutations occur and are responsible for evasion of the immune system response with high infectivity and mortality rates. While some mutations do not bring about a change in the infectivity or severity, others make the case for an investigation into how mutations change the biochemical phenotypes that allow for non-compromised interaction with entry receptor, ACE2, and reduced interaction with antibodies, either natural or commercial.

### Methods

### Choice of Tools Streamlined and Leveraged for Benchmarking of CTRL-V

In developing CTRL-V, available bioinformatic software tools were used.

ESMfold (Evolutionary Scale Modeling) is a deep learning-based method developed to predict protein structure from a sequence. For the purposes of CTRL-V, this allows for updating of the structure of proteins during each mutation.

Haddock3 (High Ambiguity Driven DOCKing) is an information-driven approach for modeling biomolecular complexes. Using two disembodied proteins as input, Haddock3 returns a complex protein that tells how two of the proteins interact structurally. This allows for obtaining interaction poses between antibodies and viral proteins.

PyRosetta is a python-based protein modeling software that includes algorithms for computational modeling and analysis of protein structures. Using PyRosetta, a user can perform single point mutation to the protein and obtain the binding energy information between two proteins at specific binding poses.

ProteinMPNN (Protein Message Passing Neural Network) is a deep learning-based method developed for protein sequence design. From a given protein structure, ProteinMPNN informs a user what other sequences can likely fold into the same structure. For the purposes of CTRL-V, this allows for narrowing down the possible amino acid mutational landscape for the viral protein.

These four software tools are independent on their own and have been integrated and automated to be executed during each design cycle of CTRL-V. Beneficially, while these tools have been explored within CTRL-V, it remains purely modular in its construction permitting the user to leverage any other tool for the relevant steps including—(a) sequence design, (b) structure prediction, (c) docking, (d) sequence evaluation through energetics, and (e) acceptance and rejection criterion of a design.

### Integer Optimization Model

An integer linear optimization programming framework was developed that solves the Favorable Mutation Problem (FMP) and generates a library of viral escape sequences. Integer optimization for sequence design through structure-informed modeling has been demonstrated in prior work to hold experimental fidelity in designing enzymes for altered substrate and cofactor specificity, protein pores for altered pore size, and affinity maturation of de novo designed antibodies. CTRL-V version 1 implements such an integer optimization model towards the FMP, wherein the wild type of SARS-CoV-2 spike protein (antigen) sequence is encoded in integer format, scored for mutations that enable viral escape, and returns the top-k preferred mutations as shown in the pseudocode in algorithm 1 built with Pyomo.

The interaction preference score was previously tabulated for all 400 pairs of 20 amino acids. A higher number in the table indicates a stronger repulsive force between the affiliated amino acid pairs. The integer optimization model utilizes this preference score for sequence design, i.e., identifying point mutations that allude to the objective of improving binding to the receptor simultaneously alleviating binding with the antibody (also referred to as the Favorable Mutation Problem). The distance between amino acids in the viral protein and antibody varies depending on the geometry of the interface.

Algorithm 1:
Algorithm 1: MULTI_OPTIMAL_SEARCH ( )

| | |
|---|---|
| 1. | optimal_solutions = new set ( ) |
| 2. | while size (optimal_solutions) < k do |
| 3. | optimals = OptimizationModel ( ) |
| 4. | optimal_solutions.add (optimals[0] ) |
| 5. | remove optimals [0] from feasible solution space |
| 6. | end while |
| 7. | return optimal_solutions |

The integer linear programming formulation has been explained as follows. First, define the variable m (equivalently, n and p) as amino acid type, i, j, and k are amino acid positions on the antibody, antigen, and receptor sequences, respectively.

The integer linear programming formulation is explained below. Here we define the variable m and n as the amino acid type,

$$m , n ∈ \{ A , C , D , E , F , G , H , I , K , L , M , N , P , Q , R , S , T , V , W , Y \}$$

i as the amino acid position in the antibody sequence and j as the amino acid position in the antigen sequence,

$$i ∈ \{ 1 , 2 , … , \vert Antibody sequence \vert \}$$

$$j ∈ \{ 1 , 2 , … , \vert Antigen sequence \vert \}$$

Cₘₙ: is defined as the preference score of the m^{th} and n^{th} amino acid pairs.
dᵢⱼ: as the distance between antibody amino acid in the i^{th} position and antigen amino acid in the j^{th} position.
Eₘᵢₙⱼ: as the scaled preference score of the antibody m amino acid in the i^{th} position and antigen n amino acid in the j^{th} position,

$${E}_{mnij} = \frac{{C}_{mn}}{{d}_{ij}^{2}}$$

To convert the protein sequence into integer format, we represent x as the antibody sequence and y as the antigen sequence; the variables are defined as:

$${x}_{im} = \{ \begin{matrix}1 & if the {i}^{th} antibody position is the m amino acid \\ 0 & otherwise\end{matrix}$$

$${z}_{kp} = \{ \begin{matrix}1 & if the {k}^{th} target molecule position is occupied by amino acid type p \\ 0 & otherwise\end{matrix}$$

$${y}_{jn} = \{ \begin{matrix}1 & if the {j}^{th} antigen position is the n amino acid \\ 0 & otherwise\end{matrix}$$

To avoid possible artifact solutions generated by the Integer Optimization Model, the following constraints were defined:

$${\sum }_{m} {x}_{im} = 1$$

$${\sum }_{i} {\sum }_{m} {x}_{im} = \vert Antibody sequence \vert$$

$${\sum }_{n} {y}_{jn} = 1$$

$${\sum }_{j} {\sum }_{n} {x}_{jn} = \vert Antigen sequence \vert$$

The objective goal within the Integer Optimization Model is to maximize the amino acid preference score between antibody and antigen; it is defined as:

$${\sum }_{m} {\sum }_{i} {\sum }_{n} {\sum }_{j} {E}_{mnij} \times {x}_{im} \times {y}_{jn}$$

CTRL-V version 1, therefore, implements a greedy best-first search (GBFS) technique that simulates the process of viral escape. GBFS prioritizes the choice that appears to be the most promising and builds rounds of mutations each progressively improving on the objective function. We simulate the viral escape process by iteratively selecting mutations that appear to be the best at escaping antibodies. In CTRL-V, we include critical observations and parity with the true nature of viral mutation, including (a) high concentration of mutations at the RBD region for immune escape, (b) only rare instances of mutation at the same polypeptide locus on the viral spike protein in consecutive generations, (c) most mutations being neutral or deleterious towards the FMP, and (d) most stable mutations cause least impact on the viral protein structure.

### CTRL-V Version 1: Integer Programming Approach for CTRL-V

In version I, the Favorable Mutation Problem was addressed through the integer programming to predict the viral escape sequences. This workflow (FIG. 2) requires an antigen-antibody complex structure as input, wherefrom the inter-residue distance information at the protein-protein interface is obtained and converted into an integer representation. Using the amino acid pairwise interaction score (as described above), new sequences for the antigen (and optionally, for the antibody; reflecting simultaneous antigen escape and antibody affinity maturation) are predicted per iteration in the integer representation.

After recovering the new mutant antigen (and optionally antibody) sequence the corresponding protein structure is predicted using ESMfold. The new antigen (and antibody) structure(s) are re-docked using HADDOCK3 to get the new interaction interface geometry (inter-residue distance matrix)—specifically, the amino acid distance between the two proteins.

This workflow runs iteratively in loops and returns a list of predicted single-point mutations without revisiting the same solution (integer cuts). CTRL-V version 1, thus, simulates the viral protein escaping from the antibody without imposing if the mutated virus is favorable for entry into human cells (i.e., binding with ACE2 is not evaluated). However, subsequent versions of CTRL-V in this study describe the computational protocol for incorporating such an evaluation. It is noteworthy that CTRL-V version 1 is equipped to handle the complex tug-of-war between antigen and antibody, where the antigen mutates to lower, and the latter mutates to restore the binding affinity between them. This represents a unique execution of an integer optimization with an iteratively moving target.

### CTRL-V Version 2: PyRosetta Sequence Design and Rigid Docking Approach

While an integer programming approach guarantees a global optimum and no stochasticity across runs, it is a depth-first (greedy) search where mutations are progressively built on one best-performing mutant—it leaves room for breadth search (i.e., exploring multiple different sequence design trajectories). To this end, in version 2, we used PyRosetta (FIG. 2), to perform single-point mutation to the viral protein stochastically. Also, we reflect minimal changes to the viral backbone structure upon mutation, ensuring side chain repacking. Much like the first version, version 2 also takes antigen-antibody complex and antigen-receptor complex structure files as inputs.

CTRL-V version 2 first identifies interacting amino acids in the complex, likely to be epitopes of the viral spike protein. It passes the list of epitope positions and the two complex files to PyRosetta. PyRosetta then ranks all possible 20-point mutations in all these positions that lower binding with antibody without compromising binding with receptor (i.e., FMP). The top-ranking mutations against both antibody and receptor are selected and used to update the antigen protein structures in both complexes using PyRosetta. After the update, the workflow automatically identifies the supplemental amino acid choices in subsequent iterations. This workflow runs iteratively in a loop along multiple design trajectories and, at the end, returns a list of predicted single-point mutations. CTRL-V version 2 simulates the viral protein escaping from the antibody, ensuring that the mutated virus is favorable for entry into human cells.

### CTRL-V Version 3: Protein MPNN-Based Sequence Design and Rigid Docking Approach

Herein the ability of CTRL-V was extended to leverage AI-based sequence design using the latest diffusion model—ProteinMPNN (FIG. 2). Compared to CTRL-V version 2, this takes antigen-antibody complex and antigen-receptor complex files and first generates, using ProteinMPNN an atlas of sequence predictions for the antigen as input. CTRL-V version 3 uses the list of sequences by ProteinMPNN as the potential mutations for the viral spike protein, which do not disrupt the secondary structure of the spike RBD (antigen) and only ranks these potential mutations against the antibody and receptor. The top-ranking mutations against both antibody and receptor are selected and used to update both complexes using PyRosetta. After the update, the system again queries the list of permitted sequences from ProteinMPNN for subsequent design iterators. Contrary to version 2, a diffusion model enables the exploration of combinatoric point mutations at different antigen loci simultaneously, albeit at a higher computational cost. ProteinMPNN also allows the generation of the sequence atlas using different biophysical constraints such as—permitting the user to define any arbitrarily sized design window on the antigen protein only where mutations will be permitted while keeping other parts of the antigen unaltered. This workflow runs iteratively in a loop and, at the end, returns a library of predicted single-point mutations that satisfy the FMP.

CTRL-V version 3, with ProteinMPNN, simulates the viral spike protein sequences that (a) maintain the spike's secondary structure and (b) enable the virus to escape from the antibody while retaining favorable entry into human host. CTRL-V's utility can be generalized without any modification for a dual bait biosensor design platform, honed over three model architecture iterations with viral escape data to assess model quality, and shown to generalize in non-viral systems (Raf kinase signaling), rather than aiming to be an escape mutation predictor.

### Example 2

This example presents the results from the workflow described in Example 1, using SARS-CoV-2 as a model virus.

Use of Experimental Data from SARS-CoV-2 for Benchmarking Tool Utility

In evaluating the performance of the approaches and versions, we use experimentally confirmed crystallographic coordinates of commercially available antibodies (LY-CoV1404; and its single-chain nanobodies) bound to SARS-CoV-2 spike RBD (PDB accession id: 6MOJ). LY-CoV1404 is a principal immunoprotein ingredient in the COVID-19 vaccines and booster shots commercially available through Pfizer® and Modema®.

CTRL-V simulation trajectories were run in parallel, for the antigen-antibody (heavy, light, and both chains separately) complexes of LY-CoV1404, 7YOW RA, 7YOW RB, 7YOW RH, and 7YOW RL. Results from all these simulations were collated and analyzed.

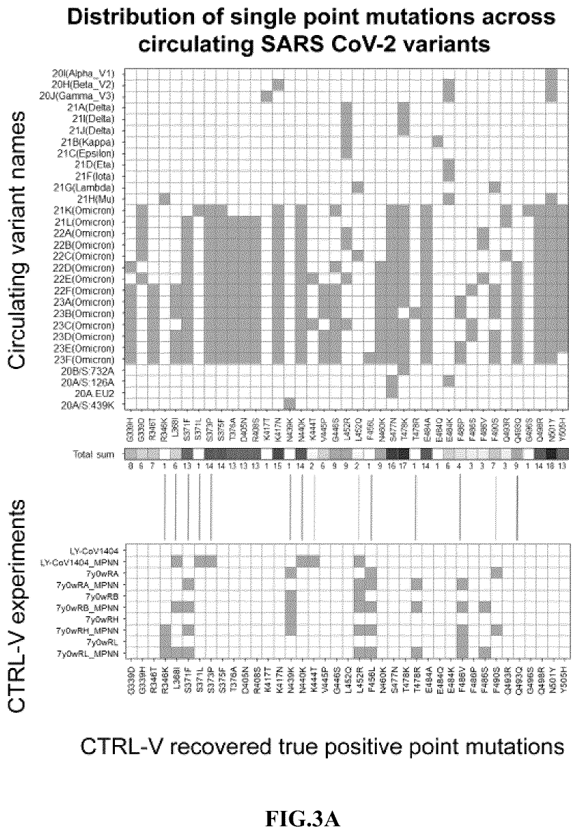

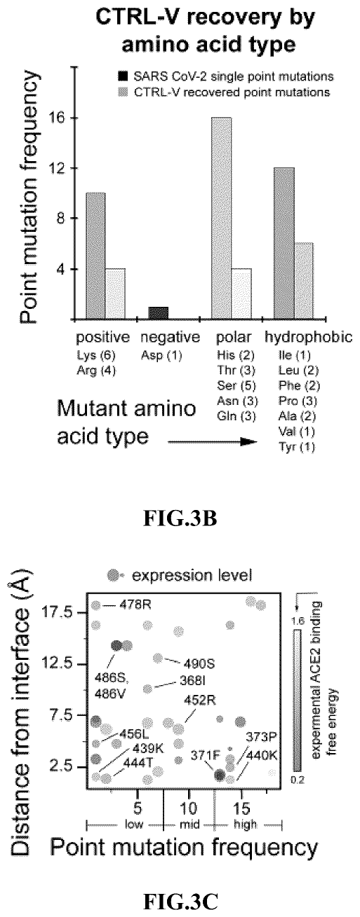

To evaluate the performance of the approaches and versions, the 30 most popular variants circulating in the last three years across the globe (as reported by Covariants, available on the world wide web at covariants.org) were identified. Among the 30 variants, there are 39 different single-point mutations, as shown in FIG. 3, which unfolds that the virus carefully screens through an exhaustive sequence landscape mutations and selects only a few specific ones that aid in the biochemical/physical objective. The objective can be, for example, to escape the antibody, survive in a specific environment, cell division, and much more. While not all mutations seen across circulating variants are a result of viral escape, this study was used to explore how many were.

The sequence of the viral spike RBD used is 195 amino acids long. Consequently, there are 20¹⁹⁵ possible mutations, and the combinatoric explosion thereof makes the computational track, even with the most powerful computing architectures today, prohibitive. Hence, instead, all possible single-point mutations were investigated, a total of 3900, with the objective to recover (reported in Table 1 as recovery rate) as many SARS-CoV-2 single-point mutations as possible using different CTRL-V versions starting from the wild-type viral antigen sequence.

While a classical definition of recovery refers to the fraction of truth set correctly identified by a computational protocol, here the truth set is the list of truly escape variants of SARS-CoV-2. Since it is so far unknown, experimentally for all 39 point mutations, whether their biochemical objective is solely antibody escape (that too from this specific LY-CoV1404 antibody), the recovery rate will be used as a computational metric to categorize how many of these point mutations are likely to be escape mutations. This then allows for the listing of potential escape mutations as per the FMP criterion. These have so far not been seen/reported in widely circulating infective strains but could be emergent in future variants. This predicted set of escape-able mutants is useful input for downstream antibody design studies.

Accuracy, Precision, Recall, and Biological Significance of these Numbers

All the approaches and versions are evaluated starting at the viral protein sequence in the antigen-antibody complex protein (Table 1). Each approach and version's performance are assessed on its accuracy, precision, and recall percentage. Due to the imbalance in true mutations and false mutations on SARS-CoV-2 single-point mutations with only 39 true mutations and 3861 false mutations (i.e., mutations that were not observed in circulating strains), the accuracy percentage has trivial significance, as a model that predicts all false mutations would have a 99% accuracy. Hence, in this experiment, we are interested in the recall percentage, which tells us how many the model can predict out of 39 circulating point mutations.

Additionally, since there is (a) non-comprehensive determination on how many of these 39 mutations arose to escape against this specific LY-CoV1404, and (b) reported polymorphisms in human ACE2 from various ecotypes (i.e., ACE2 sequences are seen to vary across geographical niches), we are using recovery rate to determine how many of these observed mutations constitute truly escape variants with respect to a specific ACE2 and antibody sequence. However, CTRL-V offers a general pipeline to simulate viral escape from any given antibody and receptor sequence.

It is hereby shown that the quality of underlying pairwise amino acid sequence interaction scores dictates the performance of the integer optimization model. By progressively finetuning the choice of appropriate tools we can segue into a powerful predictive model (Table 1).

Table 1. Approach I: CTRL-V performance (accuracy, precision, and recall) using three different versions (version 1: with integer optimization, version 2: with stochastic sequence design and docking in PyRosetta, and version 3: with AI-based diffusion model ProteinMPNN).

| | | Antibodies used (separate simulations for each) | | | | |
|---|---|---|---|---|---|---|
| | Performance | LY- | | | | |
| Method | metric | CoV1404 | 7Y0W_RA | 7Y0W_RB | 7Y0W_RH | 7Y0W_RL |
| Version 1- | Accuracy | 98.56% | 98.01% | 98.78% | 98.76% | 97.85% |
| Integer | Precision | 5.26% | 2.50% | 0.00% | 0.00% | 0.00% |
| optimization | Recall | 2.56% | 2.56% | 0.00% | 0.00% | 0.00% |
| Version 2- | Accuracy | 98.46% | 97.67% | 97.41% | 97.49% | 97.41% |
| PyRosetta | Precision | 0.00% | 5.17% | 3.03% | 1.64% | 3.03% |
| | Recall | 0.00% | 7.69% | 5.13% | 2.56% | 5.13% |
| Version 3- | Accuracy | 98.51% | 98.08% | 98.05% | 98.36% | 98.05% |
| Protein- | Precision | 19.35% | 10.87% | 15.09% | 17.95% | 15.06% |
| MPNN | Recall | 15.38% | 12.82% | 20.51% | 17.95% | 20.51% |

While version 3 is the most informative CTRL-V model, the failure of versions 1 and 2 are important to discuss and explain why each specific modification was made.

### Investigation and Explanation of CTRL-V Version 1 Performance

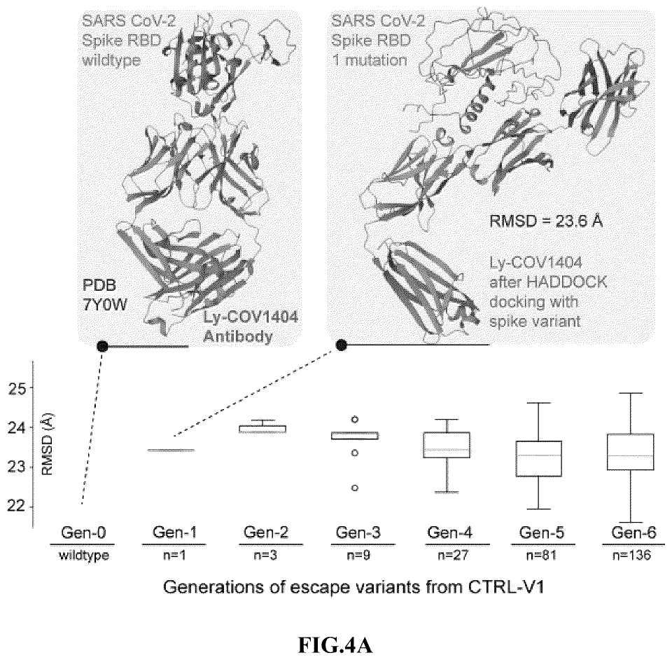

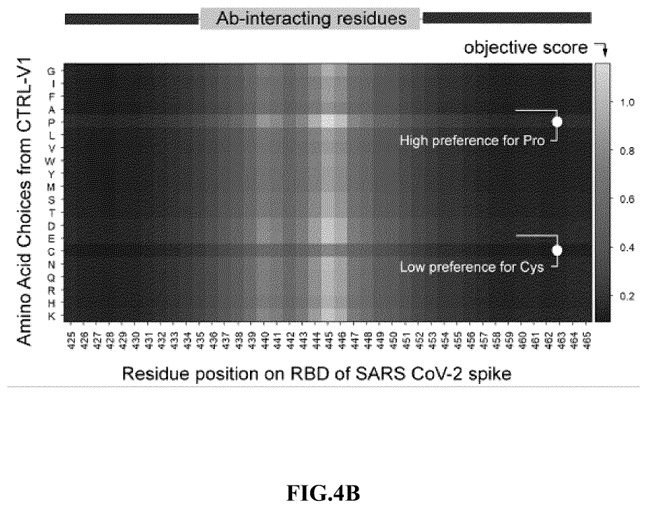

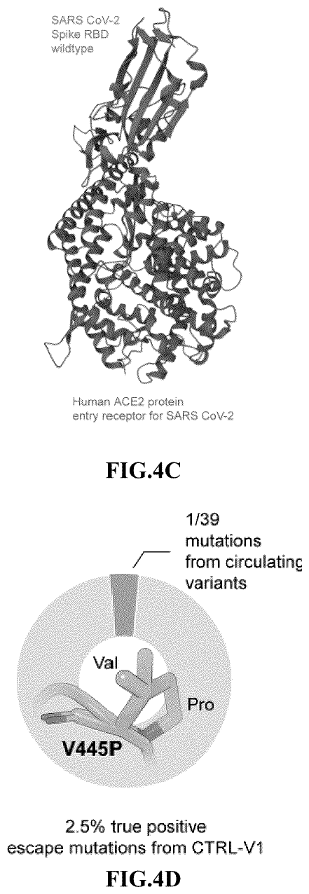

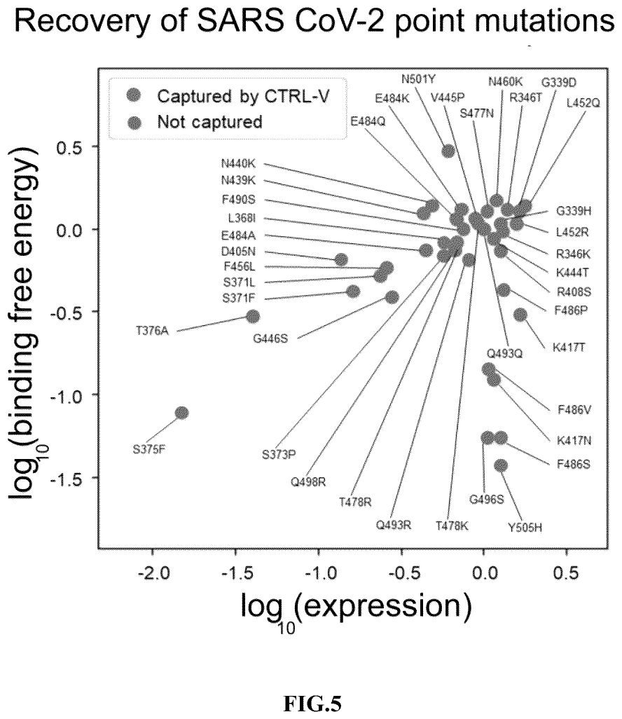

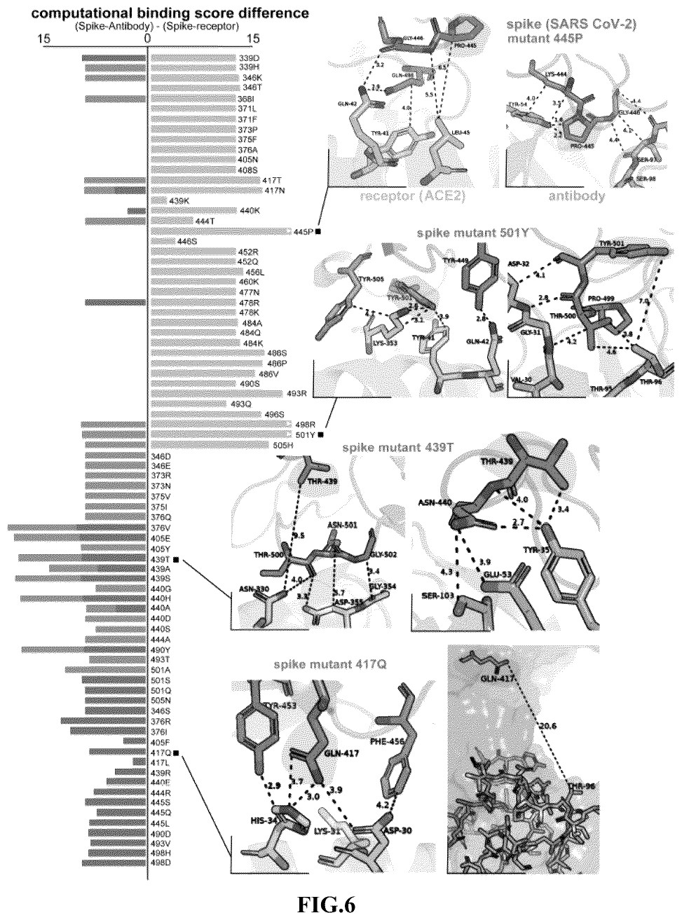

Version 1 uses an integer program to predict point mutations to the antigen (and optionally to the antibody) reliant on an experiment-derived scoring function and tools for the remaining steps: (a) ESMFold for structure prediction, and (b) HADDOCK3 for flexible docking. The prediction quality improves in recall percentage after switching from state-of-the-art tools in version 1 to a more conventional tool in version 2. From the RMSD data, (FIG. 4), it is observed that the structures are rapidly worsening with newer generations of variants. When investigating the structural prediction by ESMfold, a significant deviation (˜24 Å RMSD) in the viral protein structure is observed even with one point mutation (FIG. 4). This is expected given the poor homology across viral proteins and the low frequency of such structures in the training sets. Subsequently, HADDOCK3 reasoned over these worse, wrongly folded antigen and antibody structures and led to unreasonable results (such as splitting of heavy and light antibody chains; FIG. 4), thereby providing the integer optimization model with inaccurate distance information.

Furthermore, the amino acid preference score previously reported, albeit experimentally derived, biases the integer optimization heavily towards choosing proline, resulting in the prediction of all single-point mutations as proline. As shown in FIG. 4, a distinct horizontal patch indicates a high preference for proline and a low preference for cysteine. This is because the scores reflect that most amino acids are biochemically repelled by proline, much stronger than the strongest attractive forces, even between salt-bridging amino acids. This may call for finetuning this pairwise amino acid interaction score to account for pKa values and hydrophobicity indices of amino acids. It was decided to keep the poor performing scoring from our attempt with CTRL-V version 1, as a negative computational study as an example to show how model improvements lead to ultimately better models, often at higher computing costs.

### Inclusion of PyRosetta and Rigid Body Docking to Resolve Scoring and Handling Multichain Antibodies

As mentioned, CTRL-V version 1, utilizing Haddock3, ESMfold, and an integer optimization causes CTRL-V to be heavily biased to proline mainly due to amino acid interaction preference scoring as well as ESMfold not being catered to viral protein structure prediction. Due to that, CTRL-V version 2 was implemented with PyRosetta with rigid body docking to resolve such bias. PyRosetta's InterfaceAnalyzerMover was used for binding energy calculations and mutate_residue module for single point mutations was used for ranking each amino acid in all positions during the mutating process. This allows CTRL-V version 2 for a breadth-first search to explore all 20 amino acid choices at each polypeptide locus of the spike protein (antigen). Aside from that, with the inclusion of PyRosetta, it can be seen that the variants, even after relaxation, and energy minimization, have very similar antigen structures (RMSD <0.5 Å) for most (>93%) of the produced variants, corroborating with known experimental observation.

### Inclusion of ProteinMPNN to Explore Combinatoric Sequence Design

To assess the effect of all possible 20¹⁹⁵ single point mutations in the 195 aa residue-long RBD is computationally prohibitive for a brute force method. The addition of ProteinMPNN primarily allows for selecting potential point mutations with conserved structural information providing a robust predictive model for viral sequences. As we look closely into the predictions from CTRL-V version 2 with PyRosetta in comparison with version 3 with ProteinMPNN, the latter was able to immediately eliminate single-point mutations that seem ideal at escaping antibodies and infecting receptor but do not fold into the viral spike protein structure. For example, PyRosetta (version 2) ranks single-point mutations 4981, 501P, and 501V as the top-3 favorable mutations, while with ProteinMPNN (version 3) eliminates those mutations and selects 501A, 501S, and 444T single-point mutations. Consequently, CTRL-V version 3 shows successful recovery of known SARS-CoV-2 infective mutation, 444T, and marks it as responsible for viral escape, which is experimentally corroborated by the literature. CTRL-V-3 thus emerges as a viable dual bait biosensor design platform. This generalizability is validated by using a non-viral example (Raf kinase-Ras-Rap1a signaling pathway) where CTRL-V-3 can identify key mutational locus on Raf kinase which help it selectively bind to Ras and not Rap1a GTPase enzyme.

Biological Significance of Recovery in Context with Experimental Deep Mutational Scan Data

Looking at the performance of across three versions, CTRL-V version 3 with ProteinMPNN performed best with a little more than 20% recall percentage. While 20% does not seem like a high number when it comes to recovery of true positives by a computational algorithm, the case is more biologically nuanced here. Mutations in viruses can be ascribed to a multitude of biological factors such as survivability, cell division, environmental adaptation, and much more. Since each point mutation in a circulating variant is not necessarily indexed as solely due to escape, that too from the antibody provided in the vaccine (in isolation from the innate humoral antibody response), and a specific ACE2 ecotype—CTRL-V gives us a good indication that ˜20% ( 8/39) of viral mutations occur with the purpose of viral escape. Again, the role of the circulating variant data is to provide an anchor to optimize the right workflow for a dual bait biosensor design platform. It has been optimized through three workflow iterations spanning >5000 design trajectories, validated on known, publicly available in vivo data, and shown to work well on non-viral systems.

FIG. 5 depicts the experimental log₁₀ binding and expression scores from a deep mutational scanning of the SARS-CoV-2 receptor binding domain and unravels insights about the characteristics of the 39 infective point mutations in circulating variants. A high binding score indicates a higher binding affinity with ACE2, while a low binding score reflects weak binding. Variants with higher expression scores mean the virus expresses such variant spikes easily or more copies of the variant per unit surface area on its surface, and lower expression scores indicate poor copy numbers. The data clustering of the 39 SARS-CoV-2 single-point mutations around the origin (FIG. 5) indicates that neutral binding affinity and moderate expression score are more observed across these circulating variants. CTRL-V version 3 indicates 8/39 (20%) single-point mutations to be truly escape variants and demonstrates that the spread in their experimental expression-energy landscape is well captured by CTRL-V predictions.

### Ranking Antibody Binding Score and ACE2 Binding Score

CTRL-V predicted mutations (from version 2) were ranked as top based on computational binding affinity (using PyRosetta energies) with antibodies and ACE2 in ascending order of binding energies. A high binding score (free energy of interaction) indicates weaker binding between two proteins, and low scores indicate stronger binding. This was set up within CTRL-V version 2 (first approach), to rank single-point mutations for antigen-antibody complex interaction in descending and rank single-point mutations for antigen-receptor complex interaction in ascending. However, upon scoring the known 39 infective point mutations, it was clear that the virus does not always select amino acids with the highest binding energy against antibody and lowest binding energy against receptor (ACE2). So, we ran an additional variation of version 2 (second approach), where we predict mutations that improve binding to both ACE2 and the antibody but select the ones with the highest difference between spike-ACE2 and spike-antibody binding. Such a scoring tactic (also explored before in enzyme engineering to identify designs that improve binding to a specific substrate and eliminate binding to another) show better agreement of energy trends and recovery in viral escape variants. Without being bound by theory, it is believed that stabilizing (i.e., better interacting) mutations are always helpful for the integrity of the designed protein as much as it is for an interaction.

### Biophysical Characterization of Escape Mutations in KP.2 Variant

The SARS-CoV-2 variant KP.2, a descendant of JN.1, has emerged as a rapidly spreading lineage. A previous study investigated the virological properties of KP.2. Compared to JN.1, KP.2 possesses three substitutions: two in the S protein and one in a non-S protein. Analyses based on genome surveillance data from the USA, UK, and Canada suggest that KP.2 has a higher effective reproduction number (Re) compared to JN.1 in these regions. This indicates a potential for KP.2 to become the dominant lineage globally. While infectivity assays showed that KP.2 is significantly less infectious than JN.1, neutralization assays revealed a different picture. Sera from individuals vaccinated with the XBB.1.5 vaccine and those with breakthrough infections from various SARS-CoV-2 variants exhibited significantly lower neutralization activity against KP.2 compared to JN.1. Notably, the most significant reduction in neutralization was observed in unvaccinated individuals who received the XBB.1.5 vaccine, suggesting that KP.2 has increased immune resistance. This increased immune escape likely contributes to the higher Re of KP.2.

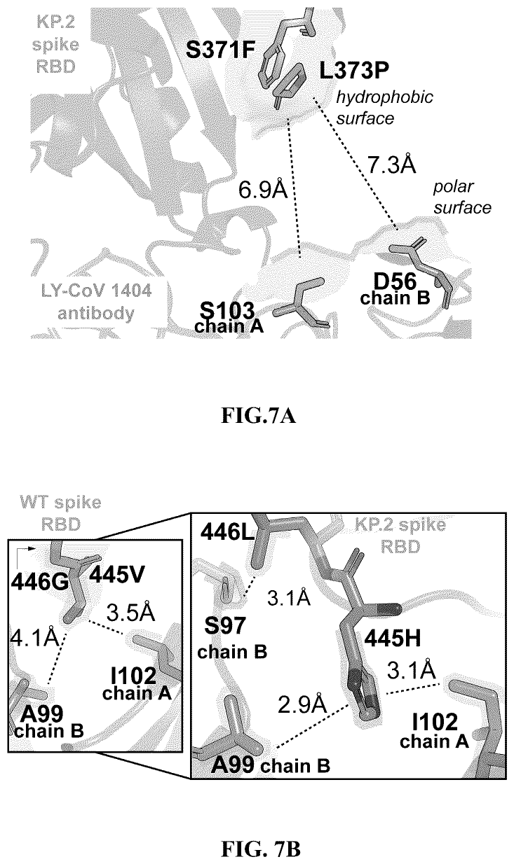

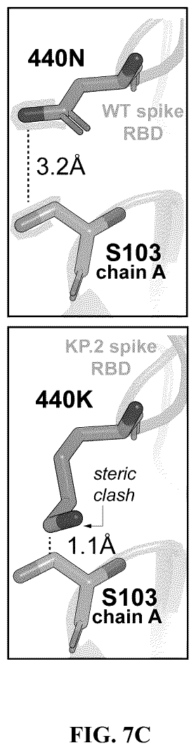

CTRL-V successfully identified five out of seven (371F, 373P, 440K, 445H, and 456L) mutations on the spike protein of KP.2 with causal biophysical underpinning that explains why these mutations enable the variant to escape the LY-CoV1404 antibody, used in COVID-19 commercial vaccine recipes. This is the first biophysical characterization of the interaction between the spike receptor binding domain of KP.2 and the LY-CoV1404 antibody, providing a molecular explanation for the reduced neutralization efficacy of this antibody against this specific variant (FIG. 7). These point mutations either introduce incompatible electrostatics or steric repulsion to reduce the LY-CoV1404 antibody's affinity for the spike protein thereby enabling viral escape. Phe371 and Pro373 mutations introduce a very hydrophobic microenvironment at ˜7 Å distance from the Ser103 (chain A), and Asp56 (chain B) of the LY-CoV1404 antibody-spike interface. His445 disrupts the hydrophobic packing at the antibody-spike complex (originally mediated by Val445) by placing a polar side chain within ˜3 Å of a hydrophobic interface domain of the antibody (Ala99 from chain A and Ile102 from chain B). Similarly, the WT SARS-CoV-2 spike uses the backbone interaction of Gly446 for electrostatic attachment with the side chain of Ser97 (chain B of antibody). However, in the KP.2 spike, Leu446 mutation creates a hydrophobic microenvironment, thereby leading to loss of attachment with the polar Ser97 side chain. Finally, mutation of 440N to 440K in KP.2 variant destroys the electrostatic contact between Asn side chain with Ser103 of antibody, by steric clashes with a 1.2 Å longer side chain conformation and 72% more bulk (80.8 and 139.1 Å² available surface areas for Asn and Lys, respectively). These mutations, recovered through CTRL-V simulations and structural information unfolds the molecular level insight about why KP.2 variant is reported to escape the administered LY-CoV1404 antibody.

### Generalization of CTRL-V Using the Raf-Ras-Rap1a Discriminatory System

To generalize the CTRL-V platform's ability to design a dual bait biosensor, the well-characterized interaction system involving Raf kinase was explored, as was its differential binding to the GTPases Ras and Rap1a Raf kinase serves as a critical node in the MAPK signaling pathway, where its ability to selectively respond to Ras but not Rap1a (despite their 56% sequence identity) plays a central role in signal transduction specificity. The molecular basis for this discrimination has been extensively characterized. While both Ras and Rap1a interact with Rafs Ras-Binding Domain (RBD) and Cysteine-Rich Domain (CRD), key differences in binding affinity and conformational effects determine signaling outcomes. This system represents an ideal test case for generalizing CTRL-V (version 3), as it exemplifies the naturally occurring phenomenon where a protein (Raf) discriminates between structurally similar partners (Ras and RapTa), preferentially binding to Ras. Using crystal structures of the Ras-Raf complex (PDB: 4GON) and the Rap1a-Raf complex (PDB: 1C1Y), CTRL-V version 3 was applied to predict mutations in the Raf binding interface, that would further enhance its discrimination between Ras and Rap1a. CTRL-V successfully identified mutations in Rafs RBD that are known to enhance its binding specificity toward Ras. Specifically, interface residue mutations Glu69, Asp66, and Glu84 were corroborated to increase the binding energy differential between Ras and Rap1a interactions. The glutamate replaces valine and establishes stronger electrostatic interactions with Ras compared to Rap1a, while the 84E mutation demonstrates how charge distribution affects binding interface complementarity.

### Example 3

The previous examples demonstrate that modular simulation platforms of the present disclosure, such as CTRL-V, offer a modular platform technology for the mechanistic delineation of viral escape mutants against a specific defense protein (antibody). The three versional implementations of CTRL-V were benchmarked against known experimental data on SARS-CoV-2 spike antigen, human ACE2 receptor, commercial LY-CoV1404 antibody, deep mutational scanning data on the antigen, and 30 circulating variants that emerged from the spike antigen. It is noteworthy that version 3 of CTRL-V recovers and identifies ˜70% (i.e., five out of seven) single point mutations that appeared in the KP.2 variant, which, as of May 2024 is responsible for 28.5% of the SARS-CoV-2 infections as per the Centers for Disease Control. Example 2 provided a detailed biophysical characterization for KP.2 escape mutations.

Although version 1, which utilizes integer optimization for sequence design and state-of-the-art tools for structure prediction and docking, yields the worst performance, we rationally discussed the reason for its poor performance. We also provide directions on how it could be improved in future versions. The version 1 has an advantage in generating overall global optimal sequences via depth-first greedy search, but it is dependent on the amino acid preference score and is vulnerable owing to its dependency on—(a) the low efficacy of the latest structure prediction tools for viral proteins, (b) inability of docking tools to handle constrained multi-chain docking tasks (where only rotamer repacking needs to be done between two chains, and flexible docking needs to be done between the third chain and the common interface of all the three chains). Version 1 has the lowest recall rate and is GPU intensive due to the ESMfold step.

Version 2 uses a more conventional sequence generation, structure updating, and docking protocols within PyRosetta, which has a moderate recall rate and is the least dependent on multiple tools interfacing with each other. However, it has a long simulation time and only produces locally optimal sequences as it stochastically explores an umbrella of sequences at each step (breadth first).

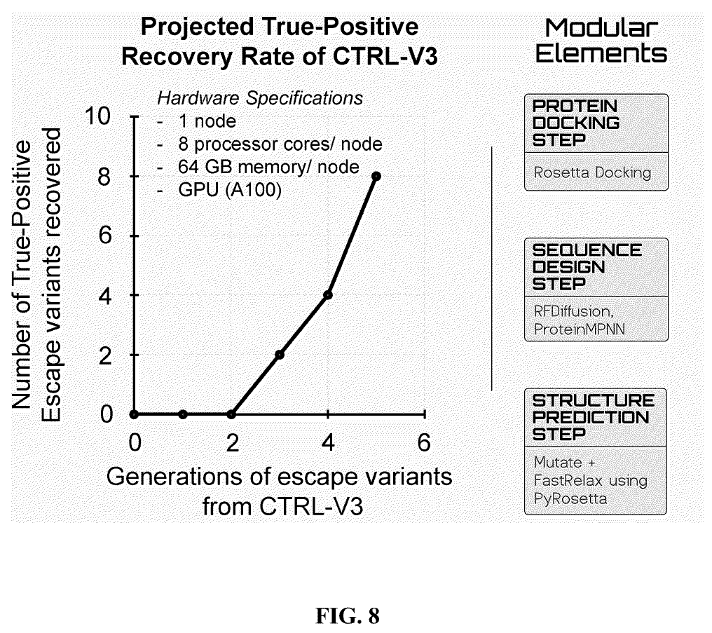

Version 3, with the addition of ProteinMPNN (AI-based diffusion model), performs best of the three and demonstrates efficacy in pinpointing select point mutations as escape variants. It has the same disadvantages as version 2 and is GPU/CPU intensive due to the ProteinMPNN. A more improved CTRL-V with a longer duration can potentially recover more single-point mutations that appear for a given antigen-antibody-receptor triad (FIG. 8 and Table 2).

Table 2. Details of 39 SARS-CoV-2 viral spike mutations as seen in circulating variants, their experimental log binding and expression data from deep mutational scanning study, computational binding energy scores (Rosetta energy function) with LY-CoV1404 antibody and human ACE2 receptor.

| | | | | ACE2 |
|---|---|---|---|---|
| Spike | Experimental | Experimental | LY-CoV1404 | binding |
| mutations | log binding | log expression | binding score | score |
| 339D | 0.11 | 0.21 | −43.23 | −29.65 |
| 339H | 0.03 | 0.1 | −43.23 | −29.65 |
| 346T | 0.12 | 0.14 | −43.76 | −29.65 |
| 346K | −0.02 | 0.12 | −43.7 | −29.65 |
| 368I | −0.08 | −0.24 | −43.23 | −29.65 |
| 371F | −0.38 | −0.79 | −43.23 | −29.65 |
| 371L | −0.28 | −0.62 | −43.23 | −29.65 |
| 373P | −0.16 | −0.24 | −43.23 | −29.65 |
| 375F | −1.11 | −1.83 | −43.23 | −29.65 |
| 376A | −0.53 | −1.4 | −43.23 | −29.65 |
| 405N | −0.19 | −0.86 | −43.23 | −29.65 |
| 408S | −0.13 | 0.1 | −43.23 | −29.65 |
| 417N | −0.91 | 0.06 | −43.23 | −25.55 |
| 417T | −0.52 | 0.22 | −43.23 | −25.76 |
| 439K | 0.09 | −0.36 | −27.13 | −29.62 |
| 440K | 0.14 | −0.31 | −43.3 | −29.65 |
| 444T | −0.06 | 0.06 | −36.46 | −29.65 |
| 445P | 0.06 | −0.05 | 209.33 | −29.59 |
| 446S | −0.41 | −0.55 | −25.76 | −29.66 |
| 452Q | 0.14 | 0.25 | −43.23 | −29.65 |
| 452R | 0.03 | 0.2 | −43.23 | −29.65 |
| 456L | −0.23 | −0.58 | −43.23 | −28.48 |
| 460K | 0.17 | 0.08 | −43.23 | −29.65 |
| 477N | 0.11 | 0.02 | −43.23 | −29.65 |
| 478K | 0.03 | −0.03 | −43.23 | −29.65 |
| 478R | −0.08 | −0.16 | −43.23 | −29.65 |
| 484A | −0.13 | −0.35 | −43.23 | −29.32 |
| 484Q | 0.06 | −0.17 | −43.23 | −29.66 |
| 484K | 0.12 | −0.13 | −43.23 | −29.07 |
| 486P | −0.37 | 0.12 | −43.23 | −25.45 |
| 486S | −1.26 | 0.1 | −43.23 | −25.08 |
| 486V | −0.85 | 0.03 | −43.23 | −29.62 |
| 490S | 0 | −0.12 | −43.23 | −29.66 |
| 493Q | 0 | 0 | −43.23 | −31.23 |
| 493R | −0.19 | −0.09 | −43.23 | −22.21 |
| 496S | −1.26 | 0.02 | −43.23 | −25.68 |
| 498R | −0.13 | −0.18 | −42.4 | −1.14 |
| 501Y | 0.47 | −0.22 | −43.23 | 13.85 |
| 505H | −1.43 | 0.1 | −43.23 | −24.54 |

In conclusion, the preference score significantly influences the predictions of CTRL-V version 1. Although version 2 can identify potentially suitable mutations, its efficacy is compromised by its inability to ascertain whether the predicted sequences will fold accurately into the viral protein structure. In CTRL-V version 3 ProteinMPNN empowers the model to make informed decisions regarding sequences that conform to the viral protein structure, yielding robust coverage of high-fidelity sequences which fold into the same structure. Due to the overall CTRL-V model being an inference model, it does not leverage any training phase; hence, it takes a long time to produce a prediction. Currently, the best model only predicts single point mutations; however, the implemented integer optimization model is best geared to account for combinatorics of point mutations (and even insertions and deletions). This can be further improved by implementing ProteinMPNN into the model to guide the choice of permitted amino acids and using the Rosetta/CHARMM/GROMACS energy functions to score the best designs.

The mutation prediction range can also be expanded outside of the binding region. Since mutations outside the binding region can also affect the structure within the binding region, molecular dynamics within the workflow can be leveraged to understand the role of long-range effects.

Ultimately, the scope and utility of the CTRL-V platform spans beyond exploring escape variants and could well be used for designing peptide-based discriminatory biosensors that progressively through design-build-test-learn iterations, reinforce from positive and negative results (i.e., machine learning), and finetunes itself to binding differentially to two different proteins, small molecules, and even metal and non-metal ions. In other words, the platform and/or workflow systems and methods disclosed herein could be used to create biosensors to determine molecules that distinguish how proteins escape vaccine protection while maintaining human binding and/or entering proteins. This would provide even additional benefits, improvements, and/or advantages.

Thus, the disclosure has been shown to include novel systems and methods for predicting escape mutations (escape variants) to be able to better plan and prepare vaccines ahead of time. For example, the iterative nature of the disclosure will allow the systems and methods to continuously and endlessly process the information of viruses to predict the next generation of strains. This will provide time for the creation of potential vaccines or other treatments that will address the mutated viruses upon actuality, which is a huge improvement. In current situations, there is generally a sense of catch-up to address the viral mutations after they appear in a host, at which point the treatments are already behind and illnesses and even death are possible until the treatments are found. Using the CTRL-V platform, groups could be simultaneously working to address existing viruses, while also planning ahead for the treatment of the most likely mutations to the viruses. Getting the treatment prepared ahead of time could reduce the severity of outbreak, and potentially save lives.

It should be noted that the CTRL-V platform is a generalized tool for viral prediction of most likely escape mutations. The tool could be used with any viruses that are attributed to any hosts. For example, the hosts could be animals (including humans), or any other host that can be affected by a virus. The identification of the escape mutations will be continuously iterated such that the identification of one or more most like escape mutations will then be re-added and operated through the system in order to determine the next, predicted iteration of the virus.

While certain examples, aspects, and/or embodiments have been disclosed, it should be appreciated that these are to be non-limiting. In addition, it should be noted that any of the aspects of any of the embodiments could be combined with other aspects in ways not explicitly disclosed herein to create even additional embodiments that would be obvious to those skilled in the art from a reading of the present disclosure. The same goes for variations, alternatives, and other changes.

1. A system for designing selective binding biosensor proteins, comprising:
a computer readable medium including steps to:
receive one or more viral inputs and output sequence design predictions using a sequence design module;
update the one or more viral inputs using a structure prediction module to create updated viral structures; and
iterate the updated viral structures via a trained protein sequence design engine to design a dual bait biosensor.

2. The system of claim 1, wherein the one or more viral inputs comprise antigen-antibody complexes and/or antigen-receptor complexes.

3. The system of claim 1, wherein the sequence design module comprises integer optimization, RosettaDesign, RFDiffusion, and/or ProteinMPNN.

4. The system of claim 1, wherein the structure prediction module comprises ESMFold, AlphaFold2, PyRosetta, and/or Biopython.

5. The system of claim 1, wherein the trained protein sequence design engine comprises ProteinMPNN.

6. The system of claim 1, wherein the one or more viral inputs are converted into an integer representation in the sequence design module.

7. The system of claim 6, further comprising the step of re-docking the updated viral structures via a protein docking module before the step of iterating the updated viral structures.

8. The system of claim 7, wherein the protein docking module comprises HADDOCK-3, SnugDock, and/or Rosetta Docking.

9. The system of claim 1, wherein the dual bait biosensor comprises proteins capable of selective binding between two proteins.

10. A method for designing a dual bait biosensor, comprising:
(a) identifying an amino acid interaction in a protein-protein complex between a first and second protein and, optionally, between a first and third protein;
(b) identifying at least one mutation in the first, second, and/or third protein that would disrupt the amino acid interaction;
(c) ranking the at least one mutation and selecting at least one favorable mutation;
(d) updating the amino acid interaction of step (a) with the favorable mutation to generate a new amino acid interaction and repeating steps (a) through (c) at least once; and
(e) designing the dual bait biosensor comprising proteins capable of selective binding between two proteins.

11. The method of claim 10, wherein step (b) comprises generating a library of sequence predictions for the first, second, and/or third proteins.

12. The method of claim 10, wherein the ranking prioritizes mutations that decrease binding affinity of the first protein to the second protein.

13. The method of claim 10, wherein the ranking deprioritizes mutations that decrease binding affinity of the first protein to the third protein.

14. The method of claim 10, wherein the first protein is an antigen, the second protein is an antibody, and/or the third protein is a receptor.

15. The method of claim 10, wherein one or more of the steps is performed using artificial intelligence.

16. The method of claim 10, wherein one or more of the steps is performed using PyRosetta and/or ProteinMPNN.

17. A method for designing a dual bait biosensor, comprising:
(a) determining an interface distance matrix of a protein-protein complex between a first and second protein;
(b) predicting a mutated protein sequence for at least the first protein using the interface distance matrix;
(c) predicting the three dimensional structure of the mutated protein sequence;
(d) predicting docking poses of the mutated protein sequence to the second protein to generate a new interface distance matrix;
(e) designing the dual bait biosensor comprising proteins capable of selective binding between two proteins.

18. The method of claim 17, wherein steps (a) through (d) are repeated at least once using the new interface distance matrix from step (d).

19. The method of claim 17, wherein step (b) is performed using integer optimization, RosettaDesign, RFDiffusion, and/or ProteinMPNN.

20. The method of claim 17, wherein step (c) is performed using ESMFold, AlphaFold2, PyRosetta, and/or Biopython.
